(19) United States
(12) Patent Application Publication
(10) Pub. No.: US 2008/0033209 A1
(43) Pub. Date: Feb. 7, 2008

(54) Method For High-Purity Quaternary Ammonium Compounds

(75) Inventors: Laszlo Szarvas, Ludwigshafen (DE); Thorsten Rohde, Mannheim (DE); Lars Wittenbecher, Mannheim (DE); Klemens Massonne, Bad Durkheim (DE)

Correspondence Address: CONNOLLY BOVE LODGE & HUTZ, LLP, P O BOX 2207, WILMINGTON, DE, 19899, US

(73) Assignee: BASF Aktiengsellschaft, Ludwigshafen (DE)

(21) Appl. No.: 11/661,080

(22) PCT Filed: Aug. 3, 2005
(86) PCT No.: PCT/EP05/08413

§ 371 Date: Mar. 23, 2007

(30) Foreign Application Priority Data
Aug. 24, 2004 (DE) 10 2004 041 139.5

(51) Int. Cl. C07C 209/20; C07D 213/20; C07D 233/54

(52) U.S. Cl. 564/282; 564/281; 564/296

(57) ABSTRACT

Process for preparing quaternary ammonium compounds by reacting the corresponding tertiary sp³-hybridized amine or sp²-hybridized imine with dimethyl sulfite, wherein the reaction is carried out
(i) in the presence of a solvent selected from the group consisting of aromatic hydrocarbons having from 6 to 10 carbon atoms, symmetrical or unsymmetrical dialkyl ethers having a total of from 5 to 10 carbon atoms, cycloalkanes having from 5 to 8 carbon atoms and C₅-C₁₀-alkanes; and (ii) at a temperature of from 10 to 100° C.

The present invention relates to a process for preparing quaternary ammonium compounds by reacting the corresponding tertiary sp³-hybridized amine or sp²-hybridized imine with dimethyl sulfite.

Quaternary ammonium compounds are important substances which are used in a wide variety of applications. Thus, they are used, for example, as active ingredients in laundry softeners, in personal hygiene products and cosmetics, as phase transfer catalysts or as electrolyte salts for electronic applications. A further important application area is ionic liquids having alkylammonium, imidazolium or pyridinium as cations.

Quaternary ammonium compounds having at least one methyl group on the nitrogen are usually prepared by alkylation of the corresponding tertiary amines with methyl esters of strong mineral acids, in particular dimethyl sulfate or methyl chloride, as methylating agents (cf., for example, Houben-Weyl, Methoden der organischen Chemie, 4th edition, volume XI/2, Georg Thieme Verlag, Stuttgart 1958, pages 591 to 630). A disadvantage of the use of dimethyl sulfate is its carcinogenic action, which represents a hazard potential and requires elaborate safety measures. Disadvantages of the use of methyl chloride are its low reactivity and consequently an increased reaction temperature and also an increased reaction pressure. This results in secondary reactions which make the work-up more difficult and reduce the yield.

As an alternative, the use of dimethyl carbonate as methylating agent is described in JP 04-341,593 and JP 09-025,173. Disadvantages of this are its low reactivity and consequently an increased reaction temperature of over 100° C. and also an increased reaction pressure of from about 1 to 4 MPa abs. This results in secondary reactions which make the work-up more difficult and reduce the yield. Thus, for example, when imidazole is methylated under these conditions, carboxylation of the ring occurs. When tertiary alkylamines are used as starting materials, the Hoffmann degradation takes place under these conditions.

Furthermore, methyl iodide is also known as methylating agent for the preparation of quaternary ammonium compounds. However, a disadvantage of the use of methyl iodide is its carcinogenic action which represents a hazard potential and requires elaborate safety measures. Furthermore, methyl iodide is not available in the required industrial amounts or is relatively expensive compared to the abovementioned methylating agents.

The use of dimethyl sulfite as methylating agent for the preparation of quaternary ammonium compounds is also known per se. Thus, the DE patent 228 247 describes the reaction of various alkaloids of the morphine group with dimethyl sulfite in the presence of methanol as solvent by heating on a water bath to form the corresponding morphinium methylsulfites (described as “methylatesulfites” in the old nomenclature used in the DE text). Chloroform and nitrobenzene are also mentioned as suitable alternative solvents. Isolation of the morphinium methylsulfites was carried out by distilling off the solvent and excess dimethyl sulfite under reduced pressure and subsequent drying. DE 228 247 also discloses the subsequent reaction of the morphinium methylsulfites obtained with metal halides or hydrohalic acids to give the corresponding morphinium halides.

JP 2001-322,970 describes the reaction of aliphatic trialkylamines with dimethyl sulfite in the presence of a polar solvent such as an alcohol or acetonitrile at from 40 to 100° C. to give the corresponding methyltrialkylammonium methylsulfites. The product was isolated by distilling off the solvent under reduced pressure. JP 2001-322,970 also discloses the subsequent reaction of the methyltrialkylammonium methylsulfites obtained with aqueous acid for the purpose of introducing the desired anion.

Compared to the other methylating agents listed above, dimethyl sulfite has the great advantage of a sufficient methylation strength which makes mild reaction conditions possible and at the same time the relative ease with which most of the methylsulfite anion can be removed by heating after addition of the acid of the desired anion to form methanol and volatile sulfur dioxide. However, it was recognized according to the invention that the processes described in DE 228 247 and JP 2001-322,970 nevertheless leave a sulfur content of the order of ≧2% by weight in the isolated quaternary ammonium compound after reaction with the acid of the desired anion. This sulfur content interferes in various applications of the quaternary ammonium compound, in particular in its use in the electronics industry. The quaternary ammonium compounds prepared by the processes described in the prior art therefore have to be firstly subjected to costly purification before use, which represents a decisive disadvantage.

It was an object of the present invention to find a process for preparing quaternary ammonium compounds which does not have the disadvantages of the prior art, is simple to carry out, in which the alkylating agent to be used is nontoxic or only slightly toxic and which makes it possible for the desired anion to be introduced simply and flexibly. Both the direct alkylation product and the product after introduction of the desired anion should be able to be prepared in high purity without complicated purification steps and should also be suitable for use in the electronics industry.

We have accordingly found a process for preparing quaternary ammonium compounds by reacting the corresponding tertiary sp³-hybridized amine or sp²-hybridized imine with dimethyl sulfite, wherein the reaction is carried out
(i) in the presence of a solvent selected from the group consisting of aromatic hydrocarbons having from 6 to 10 carbon atoms, symmetrical or unsymmetrical dialkyl ethers having a total of from 5 to 10 carbon atoms, cycloalkanes having from 5 to 8 carbon atoms and C₅-C₁₀-alkanes; and
(ii) at a temperature of from 10 to 100° C.

A property which is common to all of the solvents to be used according to the invention is their relatively low polarity, in particular in comparison with the solvents described in the prior art, for instance acetonitrile and alcohol in JP 2001-322,970 or methanol, chloroform and nitrobenzene in DE 228 247. This relatively low polarity leads to the quaternary ammonium methylsulfite formed during the reaction forming a separate solid or liquid phase and, for example, unreacted starting material or possible by-products thus remaining preferentially in the solvent phase.

Furthermore, it has surprisingly been found that the use of the solvents used according to the invention in combination with the temperature range according to the invention results, in contrast to the solvents described in the prior art, in the rearrangement of the methylsulfite anion to the methanesulfonate anion being significantly suppressed or even virtually completely prevented.

Aromatic hydrocarbons having from 6 to 10 carbon atoms which are used are generally unsubstituted benzenes or benzenes substituted by C₁-C₄-alkyl, —CH═CH—CH═CH—, 1,4-butylene, —O—CH₂—CH₂—CH₂— and also monohydroxyalkylbenzenes or monoalkoxyalkylbenzenes having a number of carbon atoms in the range specified. Examples of suitable hydrocarbons having from 6 to 10 carbon atoms are benzene, toluene, ethylbenzene, 1-propylbenzene, 2-propylbenzene, 1-butylbenzene, 2-butylbenzene, tert-butylbenzene, xylene (o-, m-, p-), methylethylbenzene (o-, m-, p-), diethylbenzene (o-, m-, p-), trimethylbenzene (vic-, sym-, asym-), cresol (o-, m-, p-), ethylphenol (o-, m-, p-), 1,2,3,4-tetrahydronaphthalene.

Symmetrical or unsymmetrical dialkyl ethers having a total of from 5 to 10 carbon atoms which are used are generally dialkyl ethers having unbranched or branched alkyl groups, with at least one alkyl group being a C₃-C₉-alkyl group. The number of carbon atoms in the other alkyl group is determined by the specified total number of carbon atoms. Examples of suitable symmetrical or unsymmetrical dialkyl ethers having a total of from 5 to 10 carbon atoms are diisopropyl ether, methyl tert-butyl ether, di-n-butyl ether and diethylene glycol dimethyl ether.

Cycloalkanes having from 5 to 8 carbon atoms which are used are generally unsubstituted or C₁-C₃-alkyl-substituted cycloalkanes. Examples of suitable cycloalkanes having from 5 to 8 carbon atoms are cyclopentane, methylcyclopentane, cyclohexane, methylcyclohexane, cycloheptane, cyclooctane.

C₅-C₁₀-alkanes used are generally unbranched or branched alkanes. Examples of suitable C₅-C₁₀-alkanes are n-pentane, 2-methylbutane (isopentane), 2,2-dimethyl-propane, n-hexane, 2-methylpentane, 3-methylpentane, 2,3-dimethylbutane, 2,2-dimethylbutane, n-heptane, isomeric heptanes, n-octane, isomeric octanes, n-nonane, isomeric nonanes, n-decane, isomeric decanes.

It is naturally also possible to use mixtures of various solvents.

Preference is given to using toluene, xylene, ethylbenzene, diethylbenzene, methyl tert-butyl ether, cyclohexane, hexane, heptane or octane as solvent in the process of the invention.

The amount of solvent used in the process of the invention is generally from 10 to 1000% by weight, preferably from 20 to 500% by weight and particularly preferably from 20 to 200% by weight, based on the amount of the tertiary sp³-hybridized amine or sp²-hybridized imine used.

The type and order of the addition of the individual starting materials and of the solvent is not critical in the process of the invention. Thus, for example, it is possible to introduce the tertiary sp³-hybridized amine or sp²-hybridized imine, the dimethyl sulfite and the solvent into the reaction apparatus either in succession in any order or simultaneously. It is also possible to admix the tertiary sp³-hybridized amine or sp²-hybridized imine and/or the dimethyl sulfite with part or the total amount of the solvent and only then combine the two solvent-comprising starting materials. Furthermore, it is also possible to place one of the two starting materials in the reaction vessel initially and to add the other starting material dropwise over a particular period of time ranging from a few minutes to a number of hours, with at least one of the starting materials being diluted with the solvent.

As reaction apparatuses for the process of the invention, it is in principle possible to use all reaction apparatuses which are suitable for a reaction in the liquid phase. These are, in particular, reaction apparatuses which make appropriate mixing of the liquid starting materials possible, for example stirred vessels.

The molar ratio of dimethyl sulfite to the tertiary sp³-hybridized amine or sp²-hybridized imine in the process of the invention is generally from 0.9 to 1.5, preferably from 0.9 to 1.2, particularly preferably from 0.9 to 1.1 and very particularly preferably from 0.95 to 1.05. If dimethyl sulfite is added in a slight excess, then a small amount of unreacted tertiary sp³-hybridized amine or sp²-hybridized imine remains in the solvent phase and can be separated off via this from the phase comprising the reaction product. If dimethyl sulfite is added in excess, the unreacted dimethyl sulfite remains in the solvent phase and can likewise be separated off via this from the phase comprising the reaction product.

The reaction between the tertiary sp³-hybridized amine or sp²-hybridized imine and the dimethyl sulfite in the process of the invention is carried out at a temperature of from 10 to 100° C. and a pressure of from 0.05 to 2 MPa abs, preferably from 0.09 to 0.5 MPa abs, particularly preferably from 0.09 to 0.2 MPa abs and very particularly preferably from 0.095 to 0.12 MPa abs.

The time required for the reaction is dependent first and foremost on the chemical nature of the starting material (reactivity of the tertiary sp³-hybridized amine or sp²-hybridized imine) and the reaction temperature selected. It can be determined, for instance, by means of preliminary experiments in which, for example, the reaction kinetics are determined, the temperature curve of the exothermic reaction is measured and/or the concentration of the starting materials and product are determined by analysis. In general, the time required is in the range from a few minutes to one day, generally of the order of from 0.1 to 24 hours, preferably of the order of from 0.1 to 10 hours.

After the reaction is complete, mixing of the reaction mixture is generally stopped, so that phase separation can take place. Depending on the type of reaction apparatus, it can be advantageous to carry out the settling of the two phases in this apparatus or in a separate settling vessel. After the two phases have settled, the liquid or solid phase of the quaternary ammonium methylsulfite obtained is separated off. In general, the phase of the quaternary ammonium methylsulfite is located at the bottom and the solvent phase is located at the top.

The solvent which has been separated off can generally be recirculated and reused as solvent for the reaction in question. It may be advisable to employ measures to prevent accumulation of possible by-products in the solvent. Possible measures which may be mentioned by way of example are (i) discharge of a small part of the solvent and replacement of this by fresh solvent or (ii) distillation of at least a small part of the solvent with subsequent recirculation.

Depending on the desired purity of the quaternary ammonium methylsulfite, it can be advantageous to subject the phase which has been separated off to a subsequent purification step. If the phase of the quaternary ammonium methylsulfite is liquid at the working temperature, it can be shaken with a suitable solvent in which the quaternary ammonium methylsulfite is insoluble or only very slightly soluble. Suitable solvents for this purpose are, for example, the solvents which can be used for the reaction according to the invention or esters such as ethyl acetate. If the phase of the quaternary ammonium methylsulfite is solid at the working temperature, it can, for example, be washed with a suitable solvent in which the quaternary ammonium methylsulfite is insoluble or only very slightly soluble. Suitable solvents for this purpose are, for example, likewise the solvents which can be used for the reaction according to the invention or esters such as ethyl acetate. Furthermore, the solid quaternary ammonium methylsulfite can also be recrystallized from a suitable solvent. Suitable solvents for this purpose are solvents in which the quaternary ammonium. methylsulfite dissolves, for example, alcohols, acetonitrile, tetrahydrofuran or nitrobenzene.

Depending on the further use of the purified or unpurified quaternary ammonium methylsulfite, it can be advantageous to dry it beforehand. If drying is carried out, it is preferably carried out at a particularly mild temperature under reduced pressure to prevent decomposition of the quaternary ammonium methylsulfite and, in particular, isomerization to the quaternary ammonium methanesulfonate.

The process of the invention can be carried out batchwise, semicontinuously or continuously. When it is carried out batchwise, the starting materials and the solvent are combined and the reaction is carried out at the desired temperature. After the reaction is complete, the reaction mixture is worked up as described. When it is carried out continuously, the two starting materials are slowly fed into the reaction apparatus for them to react at the desired temperature, with the solvent being able to be added together with one of the two starting materials, divided between the two starting materials or separately. The reaction mixture is taken off continuously in an amount corresponding to the amounts of starting materials and solvent fed in and is worked up as described. The work-up itself can likewise be carried out continuously. In the case of the semicontinuous variants, at least one of the two starting materials is slowly introduced at the desired temperature, with the reaction generally occurring in parallel with the addition. After the desired amount(s) has/have been added, the reaction mixture is generally left to react further for a particular time and is subsequently worked up as described.

In the process of the invention, the tertiary sp³-hybridized amine or tertiary sp²-hybridized imine used is preferably an amine, an imidazole, a pyridine or a guanidine.

In the process of the invention, preference is given to using an amine of the general formula (I) 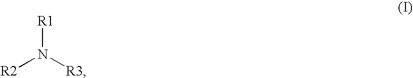
where
the radicals R¹ to R³ are each, independently of one another, a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups, with the radical R¹ also being able to be hydrogen; or
the radical R¹ is as defined above and the radicals R² and R³ together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups; or
the radicals R¹, R² and R³ together form a trivalent, carbon-comprising organic, saturated or unsaturated, acyclic or cyclic, aliphatic, aromatic or araliphatic radical which has from 1 to 40 carbon atoms and is unsubstituted or interrupted or substituted by from 1 to 5 heteroatoms or functional groups;
as tertiary sp³-hybridized amine.

In the process of the invention, preference is given to using an imidazole of the general formula (II) 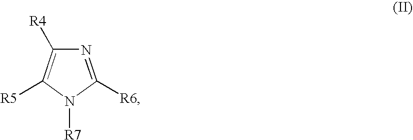
where
the radicals R⁴ to R⁷ are each, independently of one another, a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups and the radicals R⁴ to R⁶ may also be, independently of one another, hydrogen, halogen or a functional group and the radical R⁷ may also be hydrogen; or
two adjacent radicals together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups and the remaining radical is as defined above;
as tertiary sp²-hybridized imine.

In the process of the invention, preference is given to using a pyridine of the general formula (III) 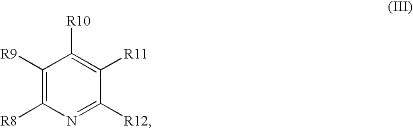
where
the radicals R⁸ to R¹² are each, independently of one another, hydrogen, halogen, a functional group or a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups; or
in each case independently, two adjacent radicals together form a divalent, carbon-comprising organic, saturated or unsaturated, acyclic or cyclic, aliphatic, aromatic or araiiphatic radical which has from 1 to 30 carbon atoms and is unsubstituted or interrupted or substituted by from 1 to 5 heteroatoms or functional groups and the remaining radicals/radical are/is as defined above;
as tertiary sp²-hybridized imine.

In the process of the invention, preference is given to using a guanidine of the general formula (IV) 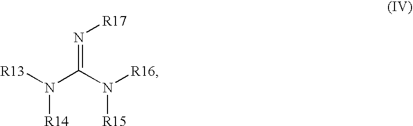
where
the radicals R¹³ to R¹⁷ are each, independently of one another, a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups, with the radicals R¹³ and R¹⁵ also being able, independently of one another, to be hydrogen; or,
in each case independently, the radicals R¹³ and R¹⁴ and/or R¹⁵ and R¹⁶ together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups and the remaining radicals/radical are/is as defined above; or
the radicals R¹⁴ and R¹⁵ together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups and the remaining radicals are as defined above;
as tertiary sp²-hybridized imine.

Possible heteroatoms are in principle all heteroatoms in the definition of the radicals R¹ to R¹⁷ which are able to formally replace a —CH₂— group, a —CH═ group, a —C— group or a ═C═ group. If the carbon-comprising radical comprises heteroatoms, then preference is given to oxygen, nitrogen, sulfur, phosphorus and silicon. Preferred groups are, in particular, —O—, —S—, —SO—, —SO₂—, —NR—, —N═, —PR—, —PR₂ and —SiR₂—, where the radicals R are the remaining part of the carbon-comprising radical. In the case of R⁴ to R⁶ and R⁸ to R¹², the carbon-comprising radical can also be bound directly via the heteroatom to the imidazolium or pyridinium ring.

Possible functional groups are in principle all functional groups which can be bound to a carbon atom or a heteroatom. Examples of suitable groups are —OH (hydroxy), ═O (in particular as a carbonyl group), —NH₂ (amino), ═NH (imino), —COOH (carboxy), —CONH₂ (carboxamide), —SO₃H (sulfo) and —CN (cyano). Functional groups and heteroatoms can also be directly adjacent, so that combinations of a plurality of adjacent atoms, e.g. —O— (ether), —S— (thioether), —COO— (ester), —CONH— (secondary amide) or —CONR— (tertiary amide), are also encompassed, for example di(C₁-C₄-alkyl)amino, C₁-C₄-alkyloxycarbonyl or C₁-C₄-alkyloxy.

As halogen, mention may be made of fluorine, chlorine, bromine and iodine.

The process of the invention is preferably carried out using amines (I), imidazoles (II), pyridines (III) and guanidines (IV) in which the radicals R⁴ to R⁶ and R⁸ to R¹² are each, independently of one another,
hydrogen;
halogen; or
a functional group;
   and the radicals R¹ to R¹⁷ are each, independently of one another,
C₁-C₁₈-alkyl which may be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups;
C₂-C₁₈-alkenyl which may be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups;
C₆-C₁₂-aryl which may be substituted by functional groups, aryl, alkyl, aryloxy, alkyloxy, halogen, heteroatoms and/or heterocycles;
C₅-C₁₂-cycloalkyl which may be substituted by functional groups, aryl, alkyl, aryloxy, alkyloxy, halogen, heteroatoms and/or heterocycles;
C₅-C₁₂-cycloalkenyl which may be substituted by functional groups, aryl, alkyl, aryloxy, alkyloxy, halogen, heteroatoms and/or heterocycles; or
a five- to six-membered, oxygen-, nitrogen- and/or sulfur-comprising heterocycle which may be substituted by functional groups, aryl, alkyl, aryloxy, alkyloxy, halogen, heteroatoms and/or heterocycles; or
   adjacent radicals R¹ and R², R² and R³, R¹ and R³, R⁴ and R⁵, R⁵ and R⁷, R⁷ and R⁶, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹³ and R¹⁷ or R¹⁶ and R¹⁷ together form
an unsaturated, saturated or aromatic ring which may be substituted by functional groups, aryl, alkyl, aryloxy, alkyloxy, halogen, heteroatoms and/or heterocycles and may be interrupted by one or more oxygen and/or sulfur atoms and/or one or more substituted or unsubstituted imino groups.

C₁-C₁₈-Alkyl which may be substituted by functional groups, aryl, alkyl, aryloxy, alkyloxy, halogen, heteroatoms and/or heterocycles is preferably methyl, ethyl, 1-propyl, 2-propyl, 1-butyl, 2-butyl, 2-methyl-1-propyl (isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl, cyclopentylmethyl, 2-cyclopentylethyl, 3-cyclopentylpropyl, cyclohexylmethyl, 2-cyclohexylethyl, 3-cyclohexylpropyl, benzyl (phenylmethyl), diphenylmethyl (benzhydryl), triphenylmethyl, 1-phenylethyl, 2-phenylethyl, 3-phenylpropyl, a,a-dimethylbenzyl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di(methoxycarbonyl)ethyl, methoxy, ethoxy, formyl, 1,3-dioxolan-2-yl, 1,3-dioxan-2-yl, 2-methyl-1 ,3-dioxolan-2-yl, 4-methyl-1,3-dioxolan-2-yl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl, 6-ethoxyhexyl, acetyl, CₙF_{2(n-a)+(1-b)}H_{2a+b} where n is from 1 to 30, 0≦a≦n and b=0 or 1 (for example CF₃, C₂F₅, CH₂CH₂—C₍ₙ₋₂₎F₂₍ₙ₋₂₎₊₁, C₆F₁₃, C₈F₁₇, C₁₀F₂₁, C₁₂F₂₅), chloromethyl, 2-chloroethyl, trichloromethyl, 1,1-dimethyl-2-chloroethyl, methoxymethyl, 2-butoxyethyl, diethoxymethyl, diethoxyethyl, 2-isopropoxyethyl, 2-butoxypropyl, 2-octyloxyethyl, 2-methoxyisopropyl, 2-(methoxycarbonyl)ethyl, 2-(ethoxycarbonyl)-ethyl, 2-(n-butoxycarbonyl)ethyl, butylthiomethyl, 2-dodecylthioethyl, 2-phenylthioethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 1 5-hydroxy-4,8,12-trioxapenta-decyl, 9-hydroxy-5-oxanonyl, 14-hydroxy-5,10-dioxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8, 12-trioxapentadecyl, 9-methoxy-5-oxanonyl, 14-methoxy-5,10-dioxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

C₂-C₁₈-alkenyl which may be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups is preferably vinyl, 2-propenyl, 3-butenyl, cis-2-butenyl, trans-2-butenyl or CₙF_{2(n-a)-(1-b)}H_{2a-b} where n≦30, 0≦a≦n and b=0 or 1.

C₆-C₁₂-aryl which may be substituted by functional groups, aryl, alkyl, aryloxy, alkyloxy, halogen, heteroatoms and/or heterocycles is preferably phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2-nitrophenyl, 4-nitrophenyl, 2,4-dinitrophenyl, 2,6-dinitrophenyl, 4-dimethylaminophenyl, 4-acetylphenyl, methoxyethylphenyl, ethoxymethylphenyl, methylthiophenyl, isopropylthiophenyl or tert-butylthiophenyl or C₆F₍₅₋ₐ₎Hₐ where 0≦a≦5.

C₅-C₁₂-cycloalkyl which may be substituted by functional groups, aryl, alkyl, aryloxy, alkyloxy, halogen, heteroatoms and/or heterocycles is preferably cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl, CₙF_{2(n-a)-(1-b)}H_{2a-b} where n≦30, 0≦a<n and b=0 or 1 or a saturated or unsaturated bicyclic system such as norbornyl or norbornenyl.

C₅-C₁₂-cycloalkenyl which may be substituted by functional groups, aryl, alkyl, aryloxy, alkyloxy, halogen, heteroatoms and/or heterocycles is preferably 3-cyclopentenyl, 2-cyclohexenyl, 3-cyclohexenyl, 2,5-cyclohexadienyl or CₙF_{2(n-a)-3(1-b)}H_{2a-3b} where n≦30, 0≦a≦n and b=0 or 1.

A five-membered to six-membered, oxygen-, nitrogen- and/or sulfur-comprising heterocycle which may be substituted by functional groups, aryl, alkyl, aryloxy, alkyloxy, halogen, heteroatoms and/or heterocycles is preferably furyl, thiophenyl, pyrryl, pyridyl, indolyl, benzoxazolyl, dioxolyl, dioxyl, benzimidazolyl, benzthioazolyl, dimethylpyridyl, methylquinolyl, dimethylpyrryl, methoxyfuryl, dimethoxypyridyl or difluoropyridyl.

If the adjacent radicals R¹ and R², R² and R³, R¹and R³, R⁴ and R⁵, R⁵ and R⁷, R⁷ and R⁶, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹³ and R¹⁷ or R¹⁶ and R¹⁷ together form an unsaturated, saturated or aromatic ring which may be substituted by functional groups, aryl, alkyl, aryloxy, alkyloxy, halogen, heteroatoms and/or heterocycles and may be interrupted by one or more oxygen and/or sulfur atoms and/or one or more substituted or unsubstituted imino groups, the two radicals together are preferably 1,3-propylene, 1,4-butylene, 1,5-pentylene, 2-oxa-1,3-propylene, 1-oxa-1,3-propylene, 2-oxa-1,3-propylene, 1-oxa-1,3-propenylene, 3-oxa-1,5-pentylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

If the abovementioned radicals comprise oxygen and/or sulfur atoms and/or substituted or unsubstituted imino groups, the number of oxygen and/or sulfur atoms and/or imino groups is not subject to any restrictions. In general, there will be no more than 5 in the radical, preferably no more than 4 and very particularly preferably no more than 3.

If the abovementioned radicals comprise heteroatoms, there is generally at least one carbon atom, preferably at least two carbon atoms, between any two heteroatoms.

The radicals R¹ to R³, R⁷ and R¹³ to R¹⁷ are particularly preferably, independently of one another, unbranched or branched C₁-C₁₂-alkyl, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, 2-hydroxyethyl, benzyl, 3-phenylpropyl, vinyl, 2-cyanoethyl, 2-(methoxycarbonyl)ethyl, 2-(ethoxycarbonyl)ethyl, 2-(n-butoxycarbonyl)ethyl, dimethylamino, diethylamino, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl, 6-hydroxyhexyl or propylsulfonic acid. In addition, particular preference is also given to the radical R⁷ being a sulfo group or an unbranched or branched sulfo-C₁-C₁₂-alkyl radical.

The radicals R⁴ to R⁶ and R⁸ to R¹² are particularly preferably, independently of one another, hydrogen or unbranched or branched C₁-C₁₂-alkyl, for example methyl, ethyl, 1-propyl, 2-propyl, 1-butyl, 2-butyl, 2-methyl-1-propyl (isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, 2-hydroxyethyl, 2-cyanoethyl, 2-(methoxycarbonyl)ethyl, 2-(ethoxycarbonyl)ethyl, 2-(n-butoxycarbonyl)ethyl, dimethylamino, diethylamino, chlorine, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl or 6-hydroxyhexyl.

Very particular preference is given to using trimethylamine, dimethylethylamine, dimethyl-n-propylamine, diethylmethylamine, triethylamine, tri-n-propylamine, di-n-propylmethylamine, tri-n-butylamine, di-n-butylmethylamine, tri-n-pentylamine, N-methylpiperidine, -dimethylaniline and N-methylmorpholine as amine (I) in the process of the invention.

Very particular preference is given to using N-methylimidazole, N-ethylimidazole, N-(1-propyl)imidazole, N-(1-butyl)imidazole, N-(1-hexyl)imidazole, N-(1-octyl)imidazole, N-(1-decyl)imidazole, N-(1-dodecyl)imidazole and N-(1-pentadecyl)imidazole as imidazole (II) in the process of the invention.

Very particular preference is given to using pyridine, 2-methylpyridine, 3-methylpyridine, 4-methylpyridine, 2,4-dimethylpyridine, 2,6-dimethylpyridine, 2-ethylpyridine and 2,6-diethylpyridine as pyridine (III) in the process of the invention.

Very particular preference is given to using N,N,N′,N′,N″-pentamethylguanidine as guanidine (IV) in the process of the invention.

If amines are used in the process of the invention, the reaction between these and the dimethyl sulfite is preferably carried out at a temperature of from 10 to 80° C., particularly preferably from 10 to 60° C. and very particularly preferably from 10 to 40° C. If imidazoles, pyridines or guanidines are used in the process of the invention, the reaction between these and the dimethyl sulfite is preferably carried out at a temperature of from 20 to 100° C., particularly preferably from 30 to 90° C. and very particularly preferably from 50 to 80° C.

If an anion other than methylsulfite is desired, the quaternary ammonium methylsulfite formed can be processed further in a further reaction step to introduce the desired anion.

A preferred anion is the hydrogensulfite anion. To obtain the quaternary ammonium hydrogensulfite, the quaternary ammonium methylsulfite formed is reacted with water to liberate methanol. As reaction apparatuses for the process of the invention, it is in principle possible to use all reaction apparatuses which are suitable for a reaction in the liquid phase. These are, in particular, reaction apparatuses which make appropriate mixing of the liquid starting materials possible, for example stirred vessels. The molar ratio of water to the quaternary ammonium methylsulfite is generally from 0.9 to 1.5, preferably from 0.95 to 1.2, particularly preferably from 0.95 to 1.1 and very particularly preferably from 0.99 to 1.05. The reaction is generally carried out at a temperature of from 10 to 80° C., preferably from 10 to 60° C. and particularly preferably from 20 to 40° C. The pressure is generally from 0.05 to 2 MPa abs, preferably from 0.09 to 0.5 MPa abs and particularly preferably from 0.095 to 0.12 MPa abs. The time required for the reaction is generally from a few minutes to a number of hours, preferably from 0.1 to 5 hours, and can, for example, be determined from the course of the reaction (pH, concentration of the methylsulfite anion). After the reaction is complete, the methanol formed and any excess water present are generally taken off under reduced pressure at a temperature of from 10 to 80° C., preferably from 10 to 60° C. The product obtained can be washed with solvents in which the quaternary ammonium hydrogensulfite is insoluble or only very sparingly soluble, for example an aromatic hydrocarbon having from 6 to 10 carbon atoms, a symmetrical or unsymmetrical dialkyl ether having a total of from 5 to 10 carbon atoms, a cycloalkane having from 5 to 8 carbon atoms or a C₅-C₁₀-alkane. It is also possible to recrystallize the product from a solvent in which the quaternary ammonium hydrogensulfite dissolves, for example an alcohol, acetonitrile, tetrahydrofuran or nitrobenzene. The product is generally dried under reduced pressure.

To introduce anions other than hydrogensulfite, the quaternary ammonium methylsulfite formed is reacted with an inorganic or organic protic acid having a pKₐ of from 1.8 to 14, measured at 25° C. in aqueous solution, to liberate methanol and sulfur dioxide and form the quaternary ammonium salt of the corresponding partially or fully deprotonated acid anion.

The pKₐ of the inorganic or organic protic acid to be used is preferably from 1.8 to 10, particularly preferably from 2 to 10 and very particularly preferably from 3 to 10, measured at 25° C. in aqueous solution. As reaction apparatuses for the process of the invention, it is in principle possible to use all reaction apparatuses which are suitable for a reaction in the liquid phase. These are, in particular, reaction apparatuses which make appropriate mixing of the liquid starting materials possible, for example stirred vessels. The molar ratio of the inorganic or organic protic acid to the quaternary ammonium methylsulfite is generally from 0.9 to 1.5, preferably from 0.95 to 1.1, particularly preferably from 0.95 to 1.05 and very particularly preferably from 0.99 to 1.02. The reaction is generally carried out at a temperature of from 10 to 80° C., preferably from 10 to 60° C. and particularly preferably from 20 to 40° C. The pressure is generally from 0.05 to 2 MPa abs, preferably from 0.09 to 0.5 MPa abs and particularly preferably from 0.095 to 0.12 MPa abs. The time required for the reaction is generally from a few minutes to a number of hours, preferably from 0.1 to 5 hours, and can, for example, be determined from the course of the reaction (pH, concentration of the methylsulfite anion). After the reaction is complete, any excess acid present is generally neutralized by means of a base, for example sodium hydroxide, and the product is subsequently washed with a solvent in which the quaternary ammonium salt dissolve does not, for example an alcohol, acetonitrile, tetrahydrofuran or nitrobenzene. The product is generally dried under reduced pressure.

The process of the invention is preferably used for preparing a quaternary ammonium salt in which the partially or fully deprotonated anion is fluoride; hexafluorophosphate; hexafluoroarsenate; hexafluoroantimonate; trifluoroarsenate; nitrite; nitrate; sulfate; hydrogensulfate; carbonate; hydrogencarbonate; phosphate; hydrogenphosphate; dihydrogenphosphate, vinylphosphonate, dicyanamide, bis(pentafluoroethyl)phosphinate, tris(pentafluoroethyl)trifluorophosphate, tris(heptafluoropropyl)trifluorophosphate, bis[oxalato(2-)]borate, bis[salicylato(2-)]borate, bis[1,2-benzenediolato(2-)-O,O′]borate, tetracyanoborate, tetracarbonylcobaltate;

tetrasubstituted borate of the general formula (Va) [BR^{a}R^{b}R^{c}R^{d}]⁻, where R^{a} to R^{d} are each, independently of one another, fluorine or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

organic sulfonate of the general formula (Vb) [R^{e}—SO₃]⁻, where R^{e} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

carboxylate of the general formula (Vc) [R^{f}—COO]⁻, where R^{f} is hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

(fluoroalkyl)fluorophosphate of the general formula (Vd) [PFₓ(C_{y}F_{2y+1-z}H_{z})₆₋ₓ]⁻, where 1≦x≦6, 1≦y≦8 and 0≦z≦2y+1;

imide of the general formulae (Ve) [R^{g}—SO₂—N—SO₂—R^{l}]⁻, (Vf) [Rⁱ—SO₂—N—CO—R^{j}]⁻ or (IVg) [R^{k}—CO—N—CO—R^{l}]⁻, where R^{g} to R^{l} are each, independently of one another, hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

methide of the general formula (Vh) 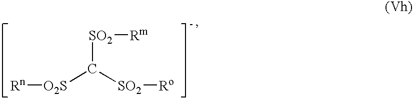
where R^{m} to R^{o} are each, independently of one another, hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

organic sulfate of the general formula (Vi) [R^{p}O—SO₃]⁻, where R^{p} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

halometalate of the general formula (Vj) [M_{q}Halᵣ]^{s-}, where M is a metal and Hal is fluorine, chlorine, bromine or iodine, q and r are positive integers and indicate the stoichiometry of the complex and s is a positive integer and indicates the charge on the complex; or

sulfide, hydrogensulfide, hydrogenpolysulfide of the general formula (Vk) [HSᵥ]⁻, polysulfide of the general formula (Vm) [Sᵥ]²⁻, where v is a positive integer from 2 to 10, thiolate of the general formula (Vn) [R^{s}S]⁻, where R^{s} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen.

Possible heteroatoms are in principle all heteroatoms which are able to formally replace a —CH₂— group, a —CH═ group, a C— group or a ═C═ group. If the carbon-comprising radical comprises heteroatoms, then preference is given to oxygen, nitrogen, sulfur, phosphorus and silicon. Preferred groups are, in particular, —O—, —S—, —SO—, —SO₂—, —NR—, —N═, —PR—, —PR₂ and —SiR₂—, where the radicals R are the remaining part of the carbon-comprising radical.

Possible functional groups are in principle all functional groups which can be bound to a carbon atom or a heteroatom. Examples of suitable groups are —OH (hydroxy), ═O (in particular as a carbonyl group), —NH₂ (amino), ═NH (imino), —COOH (carboxy), —CONH₂ (carboxamide) and —CN (cyano). Functional groups and heteroatoms can also be directly adjacent, so that combinations of a plurality of adjacent atoms, e.g. —O— (ether), —S— (thioether), —COO— (ester), —CONH— (secondary amide) or —CONR— (tertiary amide), are also encompassed.

As halogen, mention may be made of fluorine, chlorine, bromine and iodine.

Carbon-comprising organic, saturated or unsaturated, acyclic or cyclic, aliphatic, aromatic or araliphatic radicals having from 1 to 30 carbon atoms as the radicals R^{a} to R^{d} in the tetra-substituted borate (Va), the radical R^{e} in the organic sulfonate (Vb), the radical R^{f} in the carboxylate (Vc), the radicals R^{g} to Rⁱ in the imides (Ve), (Vf) and (Vg), the radicals R^{m} to R^{o} in the methide (Vh), the radical R^{p} in the organic sulfate (Vi) and the radical R^{s} in the thiolate (Vn) are preferably, independently of one another,
C₁-C₃₀-alkyl and their aryl-, heteroaryl-, cycloalkyl-, halogen-, hydroxy-, amino-, carboxy-, formyl-, —O—, —CO—, —CO—O— or —CO—N<-substituted components, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3, 3-dimethyl-1-butyl, 2-ethyl-1-butyl, 2,3-dimethyl-2-butyl, 3, 3-dimethyl-2-butyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl, hexacosyl, heptacosyl, octacosyl, nonacosyl, triacontyl, phenylmethyl (benzyl), diphenylmethyl, triphenylmethyl, 2-phenylethyl, 3-phenylpropyl, cyclopentylmethyl, 2-cyclopentylethyl, 3-cyclopentylpropyl, cyclohexylmethyl, 2-cyclohexylethyl, 3-cyclohexylpropyl, methoxy, ethoxy, formyl, acetyl or CₙF_{2(n-a)+(1-b)}H_{2a+b} where n≦30, 0≦a≦n and b=0 or 1 (for example CF₃, C₂F₅, CH₂CH₂—C₍ₙ₋₂₎F₂₍ₙ₋₂₎₊₁, C6F₁₃, C₈F₁₇, C₁₀F₂₁, C₁₂F₂₅);
C₃-C₁₂-cycloalkyl and their aryl-, heteroaryl-, cycloalkyl-, halogen-, hydroxy-, amino-, carboxy-, formyl-, —O—, —CO— or —CO—O-substituted components, for example cyclopentyl, 2-methyl-1-cyclopentyl, 3-methyl-1-cyclopentyl, cyclohexyl, 2-methyl-1-cyclohexyl, 3-methyl-1-cyclohexyl, 4-methyl-1-cyclohexyl or CₙF_{2(n-a)-(1-b)}H_{2a-b} where n≦30, 0≦a≦n and b=0 or 1;
C₂-C₃₀-alkenyl and their aryl-, heteroaryl-, cycloalkyl-, halogen-, hydroxy-, amino-, carboxy-, formyl-, —O—, —CO— or —CO—O-substituted components, for example 2-propenyl, 3-butenyl, cis-2-butenyl, trans-2-butenyl or CₙF_{2(n-a)-(1-b)}H_{2a-b} where n≦30, 0≦a<n and b=0 or 1;
C₃-C₁₂-cycloalkenyl and their aryl-, heteroaryl-, cycloalkyl-, halogen-, hydroxy-, amino-, carboxy-, formyl-, —O—, —CO— or —CO—O-substituted components, for example 3-cyclopentenyl, 2-cyclohexenyl, 3-cyclohexenyl, 2,5-cyclohexadienyl or CₙF_{2(n-a)-3(1-b)}H_{2a-3b} where n≦30, 0≦a≦n and b=0 or 1; and
aryl or heteroaryl having from 2 to 30 carbon atoms and their alkyl-, aryl-, heteroaryl-, cycloalkyl-, halogen-, hydroxy-, amino-, carboxy-, formyl-, —O—, —CO— or —CO—O-substituted components, for example phenyl, 2-methylphenyl (2-tolyl), 3-methylphenyl (3-tolyl), 4-methylphenyl, 2-ethylphenyl, 3-ethylphenyl, 4-ethylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 4-phenylphenyl, 1-naphthyl, 2-naphthyl, 1-pyrrolyl, 2-pyrrolyl, 3-pyrrolyl, 2-pyridinyl, 3-pyridinyl, 4-pyridinyl or C₆F₍₅₋ₐ₎Hₐ where 0≦a≦5.

When the anion is a tetrasubstituted borate (Va) [BR^{a}R^{b}R^{c}R^{d}]⁻, then all four radicals R^{a} to R^{d} in this are preferably identical and are preferably fluorine, trifluoromethyl, pentafluoroethyl, phenyl, 3,5-bis(trifluoromethyl)phenyl. Particularly preferred tetrasubstituted borates (Va) are tetrafluoroborate, tetraphenylborate and tetra[3,5-bis(trifluoromethyl)phenyl]borate.

When the anion is an organic sulfonate (Vb) [R^{e}—SO₃]⁻, then the radical R^{e} is preferably methyl, trifluoromethyl, pentafluoroethyl, p-tolyl or C₉F₁₉. Particularly preferred organic sulfonates (Vb) are trifluoromethanesulfonate (triflate), methanesulfonate, p-toluenesulfonate, nonadecafluorononanesulfonate (nonaflate), dimethylene glycol monomethyl ether sulfate and octylsulfate.

When the anion is a carboxylate (Vc) [R^{f}—COO]⁻, then the radical R^{f} is preferably hydrogen, trifluoromethyl, pentafluoroethyl, phenyl, hydroxyphenylmethyl, trichloromethyl, dichloromethyl, chloromethyl, trifluoromethyl, difluoromethyl, fluoromethyl, ethenyl (vinyl), 2-propenyl, —CH═CH—COO—, cis-8-heptadecenyl, —CH₂—C(OH)(COOH)—CH₂—COO⁻ or unbranched or branched C₁-C₁₈-alkyl, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, heptadecyl. Particularly preferred carboxylates (Vc) are formate, acetate, propionate, butyrate, valerate, benzoate, mandelate, trichloroacetate, dichloroacetate, chloroacetate, trifluoroacetate, difluoroacetate, fluoroacetate.

When the anion is a (fluoroalkyl)fluorophosphate (Vd) [PFₓ(C_{y}F_{2y+1-z}H_{z})₆₋ₓ]⁻, then z is preferably 0. Particular preference is given to (fluoroalkyl)fluorophosphates (Vd), in which z=0, x=3 and 1≦y≦4, specifically [PF₃(CF₃)₃]⁻, [PF₃(C₂F₅)₃]⁻, [PF₃(C₃F₇)₃]⁻ and [PF₃(C₄F₇)₃]⁻.

When the anion is an imide (Ve) [R^{g}—SO₂—N—SO₂—R^{j}]⁻, (Vf) [Rⁱ—SO₂—N—CO—R^{j}]⁻ or (Vg) [R^{k}—CO—N—CO—Rⁱ]⁻, then the radicals R^{g} to Rⁱ are each preferably, independently of one another, trifluoromethyl, pentafluoroethyl, phenyl, trichloromethyl, dichloromethyl, chloromethyl, trifluoromethyl, difluoromethyl, fluoromethyl or unbranched or branched C₁-C₁₂-alkyl, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl or dodecyl. Particularly preferred imides (Ve), (Vf) and (Vg) are [F₃C—SO₂—N—SO₂—CF₃]⁻ (bis(trifluoromethylsulfonyl)imide), [F₅C₂—SO₂—N—SO₂—C₂F₅]⁻ (bis(pentafluoroethylsulfonyl)imide), [F₃C—SO₂—N—CO—CF₃]⁻, [F₃C—CO—N—CO—CF₃]⁻ and those in which the radicals R^{g} to Rⁱ are each, independently of one another, methyl, ethyl, propyl, butyl, phenyl, trichloromethyl, dichloromethyl, chloromethyl, trifluoromethyl, difluoromethyl or fluoromethyl.

When the anion is a methide (Vh) 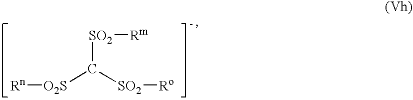
then the radicals R^{m} to R^{o} are each preferably, independently of one another, trifluoromethyl, pentafluoroethyl, phenyl, trichloromethyl, dichloromethyl, chloromethyl, trifluoromethyl, difluoromethyl, fluoromethyl or unbranched or branched C₁-C₁₂-alkyl, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-l-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl or dodecyl. Particularly preferred methides (Vh) are [(F₃C—SO₂)₃C]⁻ (tris(trifluoromethylsulfonyl)methide), [(F₅C₂—SO₂)₃C]⁻ (bis(pentafluoroethylsulfonyl)-methide) and those in which the radicals R^{m} to R^{o} are each, independently of one another, methyl, ethyl, propyl, butyl, phenyl, trichloromethyl, dichloromethyl, chloromethyl, trifluoromethyl, difluoromethyl or fluoromethyl.

When the anion is an organic sulfate (Vi) [R^{p}O—SO₃]⁻, then the radical R^{p} is preferably a branched or unbranched C₁-C₃₀-alkyl radical. Particularly preferred organic sulfates (Vi) are methylsulfate, ethylsulfate, propylsulfate, butylsulfate, pentylsulfate, hexylsulfate, heptylsulfate or octylsulfate.

When the anion is a halometalate (Vj) [M_{q}Halᵣ]^{s-}, then M is preferably aluminum, zinc, iron, cobalt, antimony or tin. Hal is preferably chlorine or bromine and very particularly preferably chlorine. q is preferably 1, 2 or 3 and r and s are determined by the stoichiometry and charge on the metal ion.

When the anion is a thiolate (Vn) [R^{s}S]⁻, then the radical Rs is preferably a branched or unbranched C₁-C₃₀-alkyl radical. Particularly preferred thiolates (Vn) are methylsulfide, ethylsulfide, n-propylsulfide, n-butylsulfide, n-pentylsulfide, n-hexylsulfide, n-heptylsulfide, n-octylsulfide or n-dodecylsulfide.

The quaternary ammonium compound prepared in the process of the invention is very particularly preferably a quaternary ammonium salt in which the partially or fully deprotonated anion is tetrafluoroborate, hexafluorophosphate, trifluoromethanesulfonate, methanesulfonate, formate, acetate, mandelate, nitrate, nitrite, trifluoroacetate, sulfate, hydrogensulfate, methylsulfate, ethylsulfate, propylsulfate, butylsulfate, pentylsulfate, hexylsulfate, heptylsulfate, octylsulfate, phosphate, dihydrogenphosphate, hydrogenphosphate, propionate, tetrachloroaluminate, Al₂Cl₇⁻, chlorozincate, chloroferrate, bis(trifluoromethylsulfonyl)imide, bis(pentafluoroethylsulfonyl)imide, tris(trifluoromethylsulfonyl)methide, bis(pentafluoroethylsulfonyl)methide, p-toluenesulfonate, bis[salicylato(2-)]borate, tetracarbonylcobaltate, dimethylene glycol monomethyl ether sulfate, octylsulfate, oleate, stearate, acrylate, methacrylate, maleate, hydrogencitrate, vinylphosphonate, bis(pentafluoroethyl)phosphinate, bis[oxalato(2-)]borate, bis[1,2-benzenediolato(2-)-O,O′]borate, dicyanamide, tris(pentafluoroethyl)trifluorophosphate, tris(heptafluoropropyl)trifluorophosphate, tetracyanoborate or chlorocobaltate.

In a general embodiment, the appropriate tertiary sp³-hybridized amine or sp²-hybridized imine, the solvent and the dimethyl sulfite are combined and this mixture is reacted while mixing, for example while stirring, at the desired temperature and the desired pressure. After the reaction is complete, mixing is stopped so that the two phases separate. The two phases are then separated from one another. The liquid or solid phase comprising the quaternary ammonium methylsulfite is preferably washed with a suitable solvent and subsequently dried under reduced pressure. The quaternary ammonium methylsulfite obtained has a high purity and can, if required, be used for preparing quaternary ammonium salts of other anions.

In a preferred embodiment, the quaternary ammonium methylsulfite obtained is reacted while mixing, for example while stirring, with an inorganic or organic protic acid having a pKₐ of ≦14, measured at 25° C. in aqueous solution, to liberate methanol and sulfur dioxide and form the quaternary ammonium salt of the corresponding partially or fully deprotonated acid anion. After the reaction is complete, any excess acid present is neutralized by means of a base, the quaternary ammonium salt obtained is washed with a suitable solvent and is subsequently dried under reduced pressure.

The process of the invention makes it possible to prepare quaternary ammonium compounds in high purity without complicated purification steps, is simple to carry out. and, due to the use of dimethyl sulfite as methylating agent, requires no toxic substances. Owing to the inventive features of the process, rearrangement of the methylsulfite formed to methanesulfonate is virtually completely avoided or at least significantly suppressed, which is decisive in making possible the high purity in respect of the isomeric by-product methanesulfonate, too. Furthermore, due to the possible reaction of the primary reaction product quaternary ammonium methylsulfite with water or an inorganic or organic protic acid, the process of the invention makes it possible to introduce other anions and is thus very flexible in terms of the choice of the obtainable anion. The particular advantage of the use of pure quaternary ammonium methylsulfite in the further reaction with water or an inorganic or organic protic acid is the easy and complete removal of the methylsulfite anion with formation of volatile methanol, (in the case of the reaction with water to form hydrogensulfite) or with formation of volatile methanol and volatile sulfur dioxide (in the case of the reaction with an inorganic or organic protic acid). In contrast thereto, the quaternary ammonium methylsulfites prepared according to the prior art comprise significant amounts of isomeric methanesulfonate which can no longer be decomposed into volatile components. This methanesulfonate would thus remain in the reaction mixture even after introduction of the desired anion and contaminate the final product.

The quaternary ammonium compounds which can be prepared by the process of the invention can therefore also be used without problems in the electronics industry.

### EXAMPLES

### Example 1 (According to the Invention)

21.11 g (0.192 mol) of dimethyl sulfite together with 100 ml of toluene were placed in a reaction vessel and a solution of 23.8 g (0.192 mol) of N-butylimidazole in 25 ml of toluene was added. The solution was stirred at 60° C. for 15 hours. A second phase formed during the reaction due to the N,N′-butylmethylimidazolium methylsulfite formed. After stirring was stopped, two phases separated. The lower phase comprising N,N′-butylmethylimidazolium methylsulfite was separated off and shaken twice with ethyl acetate. N,N′-butylmethylimidazolium methylsulfite was subsequently dried at 40° C. under a reduced pressure of 0.3 kPa (3 mbar). The weight of product obtained was 37.5 g, corresponding to 83% of the theoretical total yield (N,N′-butylmethylimidazolium methylsulfite and N,N′-butylmethylimidazolium methanesulfonate).

The liquid product obtained was analyzed by NMR spectroscopy and identified as N,N′-butylmethylimidazolium methylsulfite:

[1H-NMR, 400 Mhz], D₂O.: 0.9 ppm (t−3H); 1.3 ppm (m−2H); 1.8 ppm (m−2H); 2.8 ppm (s−3H-methanesulfonate); 3.4 ppm (s−3H); 3.8 ppm (s−3H); 4.2 ppm (t−2H); 7.4 ppm (d−2H); 8.7 ppm (s−1H).

In a quantitative evaluation of the NMR spectrum, the ratio of the signals 2.8 ppm (3H -methanesulfonate): 3.8 ppm (3H -methyl group on the imidazolium nitrogen) indicated that the proportion of methanesulfonate formed was below the detection limit. This is 3 mol %. The purity of the N,N′-butylmethylimidazolium methylsulfite was thus >97%.

### Example 2 (Comparative Example without Solvent)

62 g (0.5 mol) of N-butylimidazole were mixed with 55 g (0.5 mol) of dimethyl sulfite and heated to 80° C. whilst stirring. The reaction was strongly exothermic. The solution was stirred for another 5 hours at 80° C., subsequently cooled to about room temperature and shaken twice with ethyl acetate. N,N′-butylmethylimidazolium methylsulfite was subsequently dried at 40° C. under a reduced pressure of 0.3 kPa (3 mbar). The weight of product obtained was 108.6 g, corresponding to 92% of the theoretical total yield (N,N′-butylmethylimidazolium methylsulfite and N,N′-butylmethylimidazolium methanesulfonate).

The liquid product obtained was analyzed by NMR spectroscopy and identified as a mixture of N,N′-butylmethylimidazolium methylsulfite and N,N′-butylmethylimidazolium methanesulfonate:

[1H-NMR, 400 Mhz], D₂O.: 0.9 ppm (t−3H); 1.3 ppm (m−2H); 1.8 ppm (m−2H); 2.8 ppm (s−3H-methanesulfonate); 3.4 ppm (s−3H); 3.8 ppm (s−3H); 4.2 ppm (t−2H); 7.4 ppm (d−2H); 8.7 ppm (s−1H).

In addition, the NMR. spectrum was evaluated quantitatively and the proportion of methanesulfonate formed was calculated as 16 mol % from the ratio of signals 2.8 ppm (3H-methanesulfonate): 3.8 ppm (3H-methyl group on the imidazolium nitrogen). The purity of the N,N′-butylmethylimidazolium methylsulfite was thus only 84%.

Even though a higher yield was achieved in comparative example 2 without the use of a solvent, the N,N′-butylmethylimidazolium methylsulfite was able to be obtained in a purity of only 84%, which corresponds to a calculated yield of N,N′-butylmethyl-imidazolium methylsulfite of only about 77%. In contrast, example 1 according to the invention gives a significantly higher purity of >97%, which corresponds to a calculated yield of N,N′-butylmethylimidazolium methylsulfite of about 80-83%.

### Example 3 (Comparative Example Using acetonitrile as Solvent)

Example 3 was carried out using a procedure which was substantially analogous to example 1 of JP 2001-322,970.

20.0 g (0.198 mol) of triethylamine, 21.8 g (0.198 mol) of dimethyl sulfite and 40 ml of acetonitrile were combined and refluxed for 2 hours under atmospheric pressure while stirring. The acetonitrile was subsequently distilled off under reduced pressure and the liquid triethylmethylammonium salt was obtained. This was dissolved in 100 ml of water and admixed with 38.0 g of 50% strength aqueous tetrafluoroboric acid, corresponding to 0.198 mol of HBF₄. This solution was heated to 70° C., with the sulfur dioxide formed being given off. After evolution of sulfur dioxide had ceased, water and methanol were distilled off under reduced pressure. The theoretical total yield was 92% (triethylmethyl-ammonium tetrafluoroborate and triethylmethylammonium methanesulfonate).

Compared to example 1 of JP 2001-322,970, in which a yield of 96% is reported, the yield in the repetition of the experiment was only 92%.

The liquid product obtained was analyzed by NMR spectroscopy and the following signals were identified:

[1H-NMR, 400 Mhz], D₂O.: 1.3 ppm (t−9H ); 2.8 ppm (s−3H-methanesulfonate); 2.9 ppm (s−3H); 3.3 ppm (q−6H).

In addition, the NMR spectrum was evaluated quantitatively and the proportion of methanesulfonate formed was calculated as 6.1 mol % from the ratio of signals 2.8 ppm (3H- methanesulfonate): 2.9 ppm (3H - methyl group on the ammonium nitrogen).

The purity of the triethylmethylammonium tetrafluoroborate was thus only 93.9%.

### Example 4 (According to the Invention)

Example 4 is based on comparative example 3 above, but with the substantial difference that, according to the invention, toluene was used as solvent, with a correspondingly altered work-up, and a lower reaction temperature was chosen.

20.0 g (0.198 mol) of triethylamine were placed in a reaction vessel at room temperature and a solution of 21.8 g (0.198 mol) of dimethyl sulfite in 30 g of toluene was added dropwise. The mixture was heated to 50° C. under atmospheric pressure and was maintained under these conditions for 12 hours whilst stirring. A second liquid phase formed during the reaction due to the triethylmethylammonium methylsulfite formed. After stirring was stopped, the two phases separated. The lower phase comprising triethylmethylammonium methylsulfite was separated off and 38.0 g of 50% strength aqueous tetrafluoroboric acid, corresponding to 0.198 mol of HBF₄, were added dropwise. This solution was heated to 70° C., with the sulfur dioxide formed being given off. After evolution of sulfur dioxide had ceased, the mixture was cooled and the product was concentrated under reduced pressure with removal of water and methanol. The yield was 33.36 g, corresponding to 85% of the theoretical total yield (triethylmethylammonium methylsulfite and triethylmethylammonium methanesulfonate).

The liquid product obtained was analyzed by NMR spectroscopy and the following signals were identified:

[1H-NMR, 400 Mhz], D₂O.: 1.3 ppm (t−9H ); 2.8 ppm (s−3H-methanesulfonate); 2.9 ppm (s−3H); 3.3 ppm (q−6H).

In addition, the NMR spectrum was evaluated quantitatively and the proportion of methanesulfonate formed was calculated as 4.6 mol % from the ratio of signals 2.8 ppm (3H-methanesulfonate): 2.9 ppm (3H-methyl group on the ammonium nitrogen). The purity of the triethylmethylammonium tetrafluoroborate was thus 95.4%.

Compared to comparative example 3 using acetonitrile as solvent, the process of the invention gave a purity which was 1.5% absolute higher (95.4% vs. 93.9%). This corresponds to a reduction in the amount of undesirable triethylmethylammonium methanesulfonate of 1.5 mol %, which corresponds to a relative reduction from 24.6% relative to 75.4% relative (4.6 mol % vs. 6.1 mol %).

In the quaternization of triethylamine and subsequent replacement of the anion to form triethylmethylammonium tetrafluoroborate, too, the process of the invention leads to a significantly purer product.

### Example 5 (According to the Invention)

14.6 g (0.2 mol) of N,N-dimethylethylamine together with 150 ml of n-heptane were placed in a reaction vessel and 22.0 g (0.2 mol) of dimethyl sulfite were added dropwise at 10° C. over a period of 10 minutes. After the dropwise addition, the solution was slowly warmed to room temperature and stirred for another 4 hours. During the reaction, a white precipitate was formed. This was filtered off with suction, washed with a little heptane and dried. The weight of product obtained was 31.5 g, corresponding to 86% of the theoretical total yield (trimethylethylammonium methylsulfite and trimethylethylammonium methanesulfonate).

The solid product obtained was analyzed by NMR spectroscopy. The 1H-NMR spectrum (400 MHz, D₂O) with signals at 1.4 ppm (t−3H), 3.3 ppm (s−3H-methylsulfite anion) and 3.4 ppm (q−2H) indicates a mixture of the desired product trimethylethylammonium methylsulfite and trimethylethylammonium hydrogensulfite which had been formed by hydrolysis due to the presence of D₂O.

After aqueous work-up of the trimethylethylammonium methylsulfite, the downstream product trimethylethylammonium hydrogensulfite was able to be isolated and identified by elemental analysis.

| | Analysis | Theory |
|---|---|---|
| Carbon [g/100 g] | 35.7 | 35.5 |
| Oxygen [g/100 g] | 28.4 | 28.4 |
| Sulfur [g/100 g] | 19.0 | 18.9 |
| Hydrogen [g/100 g] | 8.5 | 8.3 |
| Nitrogen [g/100 g] | 8.7 | 8.8 |
| Σ | 100.3 | 99.9 |

### Example 6 (According to the Invention)

49.3 g (0.21 mol) of N,N′-butylmethylimidazolium methylsulfite which had been prepared in batches according to example 1 were placed in a reaction vessel at room temperature and 13 g (0.21 mol) of acetic acid were slowly added dropwise whilst stirring. The reaction mixture was carefully placed under a reduced pressure of from 50 to 0.2 kPa abs (500 to 2 mbar abs) at from 40 to 65° C., with methanol formed being distilled off. After methanol formation and distillation were complete, the reaction mixture was heated to 140° C. and freed of sulfur dioxide under a reduced pressure of 0.3 kPa abs (3 mbar abs). The sulfur dioxide was collected in a cold trap. The yield of reaction product was 37.2 g, corresponding to 90% of the theoretical total yield.

The liquid product obtained was analyzed by NMR spectroscopy and identified as N,N′-butylmethylimidazolium acetate:

[1H-NMR, 400 Mhz], D₂O.: 0.9 ppm (t−3H); 1.3 ppm (m−2H); 1.8 ppm (m−2H); 1.9 ppm (s−3H CH₃COOhu −); 3.4 ppm (s−3H); 3.8 ppm (s−3H); 4.2 ppm (t−2H); 7.4 ppm (d−2H); 8.7 ppm (s−1H).

### Example 7 (Comparative Example Using acetonitrile as Solvent)

Example 7 was carried out using a procedure which was substantially analogous to example 1 of JP 2001-322,970, but pyridine was used in place of triethylamine.

15.66 g (0.198 mol) of pyridine, 21.8 g (0.198 mol) of dimethyl sulfite and 40 ml of acetonitrile were combined and refluxed for 2 hours under atmospheric pressure while stirring. The acetonitrile was subsequently distilled off under reduced pressure and the liquid methylpyridinium salt was obtained. This was dissolved in 100 ml of water and admixed with 38.0 g of 50% strength aqueous tetrafluoroboric acid, corresponding to 0.198 mol of HBF₄. This solution was heated to 70° C., with the sulfur dioxide formed being given off. After evolution of sulfur dioxide had ceased, water and methanol were distilled off under reduced pressure. The theoretical total yield was 86.8% (methylpyridinium methylsulfite and methylpyridinium methanesulfonate).

The liquid product obtained was analyzed by NMR spectroscopy and the following signals were identified:

[1H-NMR, 400 Mhz], D₂O.: 2.8 ppm (s−3H-methanesulfonate); 4.4 ppm (s−3H); 4.45 ppm (s−3H-secondary components); 8.0 ppm (m, 2H); 8.5 ppm (m−1H); 8.8 ppm (m−2H).

In addition, the NMR spectrum was evaluated quantitatively and the proportion of methanesulfonate formed was calculated as 10.5 mol % from the ratio of signals 2.8 ppm (3H-methanesulfonate): 4.4 ppm (3H-methyl group on the pyridinium nitrogen). The purity of the pyridinium tetrafluoroborate was thus only 89.5%.

### Example 8 (According to the Invention)

15.82 g (0.2 mol) of pyridine were placed in a reaction vessel at room temperature and a mixture of 22 g (0.2 mol) of dimethyl sulfite and 30 g of toluene was slowly added dropwise. The mixture obtained was heated to 50° C. and stirred for 12 hours. After the reaction mixture had been cooled, the lower phase comprising methylpyridinium methylsulfite was separated off and 39 g of 50% strength aqueous tetrafluoroboric acid, corresponding to 0.2 mol of HBF₄, were added dropwise to this. Gas evolution was observed. The reaction mixture was then stirred at 70° C. for 2 hours and then evaporated at 120° C. and 0.2 kPa abs (2 mbar abs). The weight of product obtained was 28.5 g, corresponding to 85% of the theoretical total yield (methylpyridinium tetrafluoroborate and methylpyridinium methanesulfonate).

The liquid product obtained was analyzed by NMR spectroscopy and the following signals were identified:

[1H-NMR, 400 Mhz], D₂O.: 2.8 ppm (s−3H, methanesulfonate); 4.4 ppm (s, 3H-methyl group on the pyridinium nitrogen); 8.1 ppm (m, 2H); 8.5 ppm (m, 1H); 8.8 ppm (m, 2H).

In a quantitative evaluation of the NMR spectrum, the ratio of signals 2.8 ppm (3H-methanesulfonate): 4.4 ppm (3H-methyl group on the pyridinium nitrogen) indicated that the proportion of methanesulfonate formed was below the detection limit. This is 3 mol %. The purity of the methylpyridinium tetrafluoroborate was thus >97%.

1-14. (canceled)

15. A process for preparing quaternary ammonium compounds comprising the step of reacting the corresponding tertiary sp³-hybridized amine or sp²-hybridized imine with dimethyl sulfite, wherein the reaction is carried out
(i) in the presence of a solvent selected from the group consisting of aromatic hydrocarbons having from 6 to 10 carbon atoms, symmetrical or unsymmetrical dialkyl ethers having from 5 to 10 carbon atoms, cycloalkanes having from 5 to 8 carbon atoms, and C₅ to C₁₀ alkanes; and
(ii) at a temperature of from 10° C. to 100° C.

16. The process according to claim 15, wherein said solvent is present in an amount of from 10 to 1000% by weight, based on the weight of said tertiary sp³-hybridized amine or sp2-hybridized imine.

17. The process according to claim 15, wherein said solvent is toluene, xylene, ethylbenzene, diethylbenzene, methyl tert-butyl ether, cyclohexane, hexane, heptane or octane.

18. The process according to claim 15, wherein the molar ratio of said dimethyl sulfite to said tertiary sp³-hybridized amine or sp²-hybridized imine is from 0.9 to 1.5.

19. The process according to claim 15, wherein the quaternary ammonium compound prepared forms a liquid or solid phase which is separated off after the reaction.

20. The process according to claim 15, wherein said tertiary sp³-hybridized amine is an amine of the general formula (I) 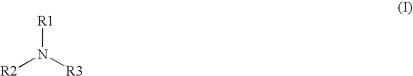
wherein
R¹, R², and R³ are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and wherein R¹ is optionally hydrogen; or
R¹ is hydrogen or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and R² and R³ together form a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; or
R¹, R², and R³ together form a trivalent, saturated or unsaturated, acyclic or cyclic, aliphatic, aromatic or araliphatic radical having up to 40 carbon atoms, wherein said trivalent radical is optionally substituted with up to 5 functional groups and wherein up to 5 of the carbon atoms of said trivalent radical is optionally replaced with heteroatoms.

21. The process according to claim 15, wherein said tertiary sp²-hybridized imine is an imidazole of the general formula (II) 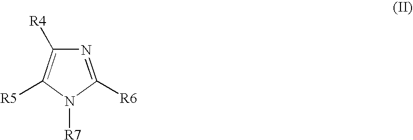
wherein
R⁴, R⁵, R⁶, and R⁷ are each, independently of one another, hydrogen, a sulfo group, or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and wherein R⁴, R⁵, and R⁶ are optionally, independently of one another, halogen; or
R⁴ and R⁵ or R⁵ and R⁶ or R⁶ and R⁷ together form a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; and the remaining substituents are each, independently of one another, hydrogen, a sulfo group, or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms, and optionally halogen when R⁴, R⁵, or R⁶ are remaining substituents.

22. The process according to claim 15, wherein said tertiary sp²-hybridized imine is a pyridine of the general formula (III) 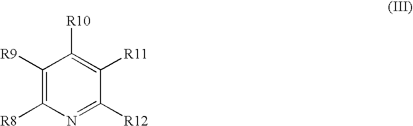
wherein
R⁸, R⁹, R¹⁰, R¹¹, and R¹² are each, independently of one another, hydrogen, halogen, a functional group or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; or
R⁸ and R⁹ or R⁹ and R¹⁰ or R¹¹ and R¹¹ or R¹¹ and R¹² together form a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; and the remaining substituents are each, independently of one another, hydrogen, halogen, a functional group or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms.

23. The process according to claim 15, wherein said tertiary sp²-hybridized imine is a guanidine of the general formula (IV) 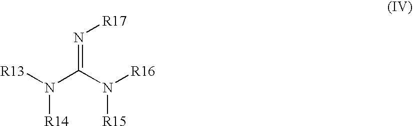
wherein
R¹³, R¹⁴, R⁵, R¹⁶, and R¹⁷ are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and wherein R¹³ and R¹⁵ are optionally, independently of one another, hydrogen; or
R¹³ and R¹⁴ and/or R¹⁵ and R¹⁶ together form, independently in each case, a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; and the remaining substituent or subtituents are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms, and optionally hydrogen when R¹³ and R¹⁵ are remaining substituents; or
R¹⁴ and R¹⁵ together form a divalent, saturated or unsaturated, acyclic or cyclic, aliphatic, aromatic or araliphatic radical having up to 30 carbon atoms, wherein said radical is optionally substituted with up to 5 functional groups and wherein up to 5 of the carbon atoms of said radical is optionally replaced with heteroatoms; and the remaining substituents are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms, and R¹³ is optionally hydrogen.

24. The process according to claim 15, wherein said tertiary sp³-hybridized amine or sp²-hybridized imine is trimethylamine, dimethylethylamine, dimethyl-n-propylamine, diethylmethylamine, triethylamine, tri-n-propylamine, di-n-propylmethylamine, tri-n-butylamine, di-n-butylmethylamine, tri-n-pentylamine, N-methylpiperidine, dimethylaniline, N-methylmorpholine, N-methylimidazole, N-ethylimidazole, N-(1-propyl)imidazole, N-(1-butyl)imidazole, N-(1-hexyl)imidazole, N-(1-octyl)imidazole, N-(1-decyl)imidazole, N-(1-dodecyl)imidazole, N-(1-pentadecyl)imidazole, pyridine, 2-methylpyridine, 3-methylpyridine, 4-methylpyridine, 2,4-dimethylpyridine, 2,6-dimethylpyridine, 2-ethylpyridine, 2,6-diethylpyridine, or N,N,N′,N′,N″-pentamethylguanidine.

25. The process according to claim 15, wherein the quaternary ammonium compound prepared is subsequently reacted with water to form a quaternary ammonium hydrogensulfite.

26. The process according to claim 15, wherein the quaternary ammonium compound prepared is reacted with an inorganic or organic protic acid having a pKa of ≦14 measured at 25° C. in aqueous solution to form a quaternary ammonium salt of said inorganic or organic protic acid, wherein the anion of said quaternary ammonium salt is partially or fully deprotonated.

27. The process according to claim 26, wherein said partially or fully deprotonated anion is
fluoride; hexafluorophosphate; hexafluoroarsenate; hexafluoroantimonate; trifluoroarsenate; nitrite; nitrate; sulfate; hydrogensulfate; carbonate; hydrogencarbonate; phosphate; hydrogenphosphate; dihydrogenphosphate; vinylphosphonate; dicyanamide; bis(pentafluoroethyl)phosphinate; tris(pentafluoroethyl)trifluorophosphate; tris(heptafluoropropyl)trifluorophosphate; bis[oxalato(2-)]borate; bis[salicylato(2-)]borate; bis[1,2-benzenediolato(2-)-O,O′]borate; tetracyanoborate; tetracarbonylcobaltate; tetrasubstituted borate of the general formula (Va)
[BR^{a}R^{b}R^{c}R^{d}]⁻ (Va)
wherein R^{a}, R^{b}, R^{c}, and R^{d} are each, independently one another, fluorine or a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally
replaced with heteroatoms;
organic sulfonate of the general formula (Vb)
[R^{e}—SO₃]⁻ (Vb)
wherein R^{e} is a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
carboxylate of the general formula (Vc)
[R^{f}—COO]⁻ (Vc)
wherein R^{f} is hydrogen or a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
(fluoroalkyl)fluorophosphate of the general formula (Vd)
[PFₓ(C_{y}F_{2y+1-z}H_{z})₆₋ₓ]⁻ (Vd)
wherein 1≦x≦6, 1≦y≦8 and 0≦z≦2y+1;
imide of the general formulae (Ve), (Vf), and (Vg)
[R^{g}—SO₂—N—SO₂—R^{h}]⁻ (Ve)
[Rⁱ—SO₂—N—CO—R^{j}]⁻ (Vf)
[R^{k}—CO—N—CO—R^{l}]⁻ (Vg)
wherein R^{g}, R^{h}, Rⁱ, R^{j}, R^{k}, and R^{l} are each, independently of one another, hydrogen or a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
methide of the general formula (Vh) 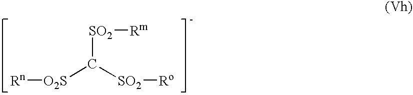
wherein R^{m}, Rⁿ, and R^{o} are each, independently of one another, hydrogen or a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
organic sulfate of the general formula (Vi)
[R^{p}O—SO₃]⁻ (Vi)
wherein R^{p} is a saturated or unsaturated, acyclic or cyclic, aliphatic, aromatic or araliphatic radical which has from 1 to 30 carbon atoms, wherein said radical is optionally substituted with one or more functional groups or halogen and wherein one or more carbon atoms of said radical is optionally replaced with heteroatoms;
halometalate of the general formula (Vj)
[M_{q}Halᵣ]^{s-} (Vj)
wherein
M is a metal
Hal is fluorine, chlorine, bromine or iodine, and
q, r, and s are positive integers; or
sulfide, hydrogensulfide, hydrogenpolysulfide of the general formula (Vk)
[HSᵥ]⁻ (Vk)
wherein v is a positive integer from 2 to 10;
polysulfide of the general formula (Vm)
[Sᵥ]²⁻ (Vm)
wherein v is a positive integer from 2 to 10;
thiolate of the general formula (Vn)
[R^{s}S]⁻ (Vn)
wherein R^{s} is a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms.

28. The process according to claim 27, wherein said partially or fully deprotonated anion is tetrafluoroborate, hexafluorophosphate, trifluoromethanesulfonate, methanesulfonate, formate, acetate, mandelate, nitrate, nitrite, trifluoroacetate, sulfate, hydrogensulfate, methylsulfate, ethylsulfate, propylsulfate, butylsulfate, pentylsulfate, hexylsulfate, heptylsulfate, octylsulfate, phosphate, dihydrogenphosphate, hydrogenphosphate, propionate, tetrachloroaluminate, Al₂Cl₇⁻, chlorozincate, chloroferrate, bis(trifluoromethylsulfonyl)imide, bis(pentafluoroethylsulfonyl)imide, tris(trifluoromethylsulfonyl)methide, bis(pentafluoroethylsulfonyl)methide, p-toluenesulfonate, bis[salicylato(2-)]borate, tetracarbonylcobaltate, dimethylene glycol monomethyl ether sulfate, octylsulfate, oleate, stearate, acrylate, methacrylate, maleate, hydrogencitrate, vinylphosphonate, bis(pentafluoroethyl)phosphinate, bis[oxalato(2-)]borate, bis[1,2-benzenediolato(2-)-O,O′]borate, dicyanamide, tris(pentafluoroethyl)trifluorophosphate, tris(heptafluoropropyl)trifluorophosphate, tetracyanoborate, or chlorocobaltate.
